(19) United States
(12) Patent Application Publication
(10) Pub. No.: US 2015/0226734 A1
(43) Pub. Date: Aug. 13, 2015

(54) INSTANT OSTEOARTHRITIS TEST PAPER

(71) Applicants: Yu Kuo, Kaohsiung City (TW); Yu-Chien Lin, Kaohsiung City (TW)

(72) Inventors: Yu Kuo, Kaohsiung City (TW); Yu-Chien Lin, Kaohsiung City (TW)

(21) Appl. No.: 14/178,679

(22) Filed: Feb. 12, 2014

(51) Int. Cl. G01N 33/543

(52) U.S. Cl. CPC G01N 33/54373

(57) ABSTRACT

An instant osteoarthritis test paper is used to detect a urine sample and comprises a substrate, a test result layer arranged on the substrate, a water absorption layer connecting with the substrate and partially covering the test result layer, an antibody release layer connecting with the substrate and partially covering the test result layer, and a sample reception layer connecting with the substrate and partially covering the antibody release layer. The test result layer includes a result indication zone and a control zone. The antibody release layer includes a plurality of test complexes each containing a colloidal gold and a plurality of polyclonal antibodies to COMP binding to the colloidal gold. The instant osteoarthritis test paper provides a non-invasive inspection method, and thereby favors early detection and therapy of degeneration of articular cartilage to slow down the deterioration speed of the disease.

### FIELD OF THE INVENTION

The present invention relates to a test device, particularly to an instant non-invasive osteoarthritis test paper.

### BACKGROUND OF THE INVENTION

Osteoarthritis (OA) is a common disease involving articular cartilage degeneration. According to statistics, 70-80% of persons aged over 65 suffer from OA.

The current technologies are not easy to detect the extent of degeneration of articular cartilage. Normally, X-ray is used to detect the gap between the articular cartilages to determine the extent of degeneration of articular cartilage. However, the current technologies, including clinic test, X-ray inspection and MRI (Magnetic Resonance Imaging) inspection, are hard to detect the early-stage degeneration of articular cartilage. While X-ray inspection or MRI inspection detects abnormal conditions, the degeneration of articular cartilage has been unable to recover. Therefore, X-ray inspection or MRI inspection cannot detect the early-stage OA. Some of ordinary preventive OA inspections need to invasively extract the tissue fluid of articular cartilage and then undertake LC/MS/MS (Liquid Chromatography/Mass Spectrometry/Mass Spectrometry) to determine the quantity of the decomposed cartilage and the extent of cartilage damage. Some of ordinary preventive OA inspections extract the synovial fluid or blood serum and then undertake ELISA (Enzyme-Linked Immunosorbent Assay) to determine the quantity of the decomposed cartilage and the extent of cartilage damage. Therefore, there has not yet been a cheaper and fast method to detect OA so far. It is estimated that 70-80% of persons aged over 65 have OA.

### SUMMARY OF THE INVENTION

The primary objective of the present invention is to solve the problem that the conventional technologies cannot detect the early-stage OA in a simple and non-invasive way.

To achieve the above-mentioned objective, the present invention proposes an instant osteoarthritis (OA) test paper, which is used to detect a urine sample, and which comprises a substrate, a test result layer arranged on the substrate, a water absorption layer connected with the substrate, an antibody release layer connected with the substrate, and a sample reception layer connected with the substrate and partially covering the antibody release layer. The test result layer includes a result indication zone and a control zone. The test result layer is divided by the result indication zone and the control zone into a test section between the result indication zone and the control zone, a first connection section arranged at one side of the control zone far away from the test section, and a second connection section arranged at one side of the result indication zone far away from the test section. The water absorption layer partially covers the first connection section. The antibody release layer partially covers the second connection section. The antibody release layer includes a plurality of test complexes each containing a colloidal gold and a plurality of polyclonal antibodies to COMP (Cartilage Oligomeric Matrix Protein) binding with the colloidal gold. The instant osteoarthritis test paper of the present invention provides a non-invasive inspection method, and thereby favors early detection and therapy of degeneration of articular cartilage to slow down the deterioration speed of the disease.

The present invention is characterized in:
1. The combination of the polyclonal antibodies to COMP in the test complexes and the COMP in the urine sample is used to facilitate early-stage detection and therapy of OA, whereby to overcome the problem that the conventional X-ray inspection can only detect OA in the later stage or when the articular cartilage has been degenerated.
2. The instant OA test paper of the present invention is a non-invasive inspection method used to examine urine samples, and thus exempts testee from discomfort caused by the conventional invasive inspection methods.

### BRIEF DESCRIPTION OF THE DRAWINGS

FIG. 1 is a perspective view schematically showing an instant OA test paper according to one embodiment of the present invention;

FIG. 2 is a sectional view schematically showing an instant OA test paper according to one embodiment of the present invention;

FIG. 3 is a top view schematically showing an instant OA test paper according to one embodiment of the present invention; and

FIGS. 4A-4C are diagrams schematically showing the test results of an instant OA test paper according to one embodiment of the present invention.

### DETAILED DESCRIPTION OF THE PREFERRED EMBODIMENTS

The technical contents of the present invention are described in detail in cooperation with drawings below.

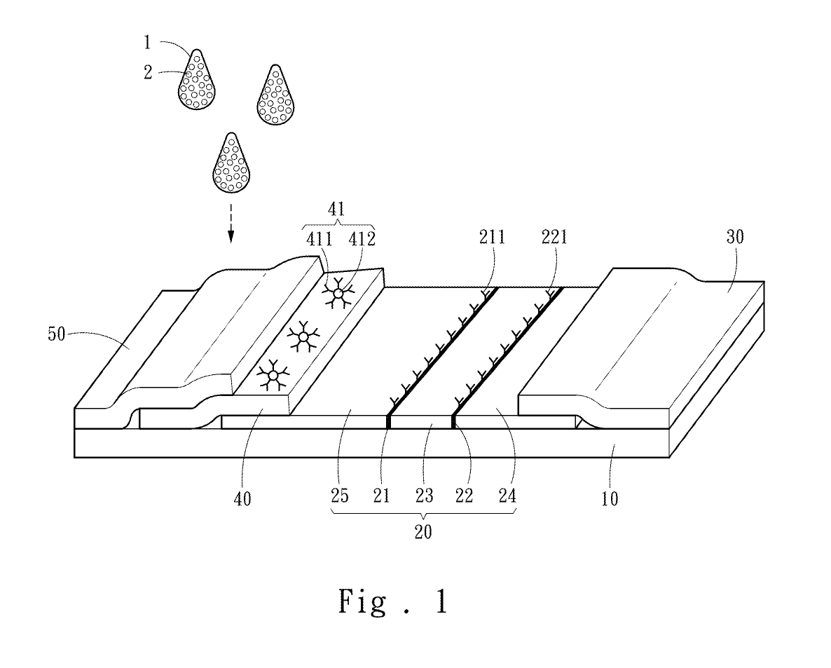

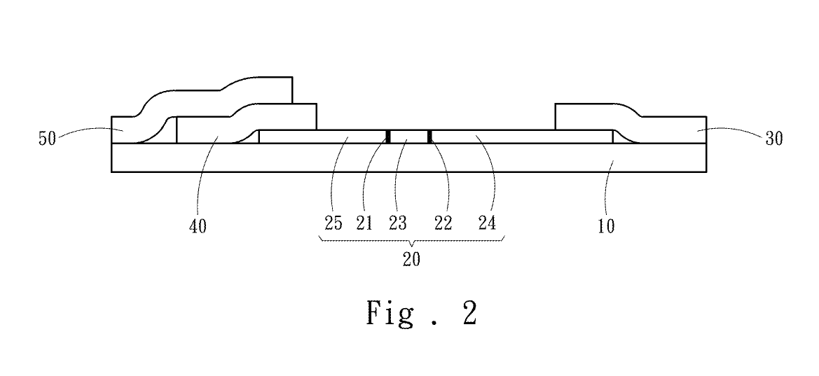

Refer to FIG. 1 and FIG. 2. The present invention proposes an instant osteoarthritis (OA) test paper, which is used to detect a urine sample, and which comprises a substrate 10, a test result layer 20 arranged on the substrate 10, a water absorption layer 30 connected with the substrate 10, an antibody release layer 40 connected with the substrate 10, and a sample reception layer 50 connected with the substrate 10 and partially covering the antibody release layer 40. The test result layer 20 is water-absorptive nitrocellulose paper. The antibody release layer 40 and the sample reception layer 50 are made of water-absorptive glass fiber. The test result layer 20 includes a result indication zone 21 and a control zone 22. The result indication zone 21 contains a plurality of monoclonal antibodies to COMP (Cartilage Oligomeric Matrix Protein) 211, and the control zone 22 contains a plurality of secondary antibodies 221. The test result layer 20 is divided by the result indication zone 21 and the control zone 22 into a test section 23 between the result indication zone 21 and the control zone 22, a first connection section 24 arranged at one side of the control zone 22 far away from the test section 23, and a second connection section 25 arranged at one side of the result indication zone 21 far away from the test section 23. The water absorption layer 30 partially covers the first connection section 24. The overlapped area of the water absorption layer 30 and the first connection section 24 has a width of 0.1-0.3 cm. The antibody release layer 40 partially covers the second connection section 25. The overlapped area of the antibody release layer 40 and the second connection section 25 also has a width of 0.1-0.3 cm. The antibody release layer 40 includes a plurality of test complexes 41 each containing a colloidal gold 412 and a plurality of polyclonal antibodies to COMP 411 binding to the colloidal gold 412.

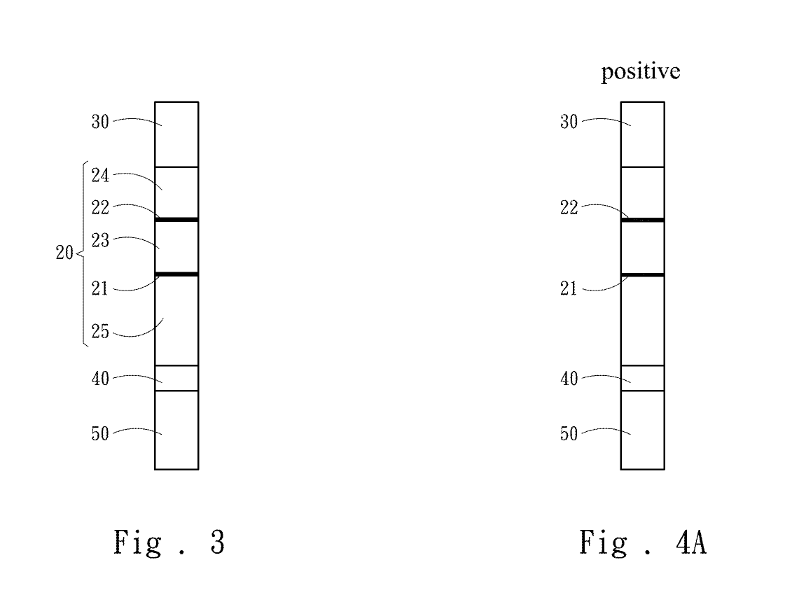

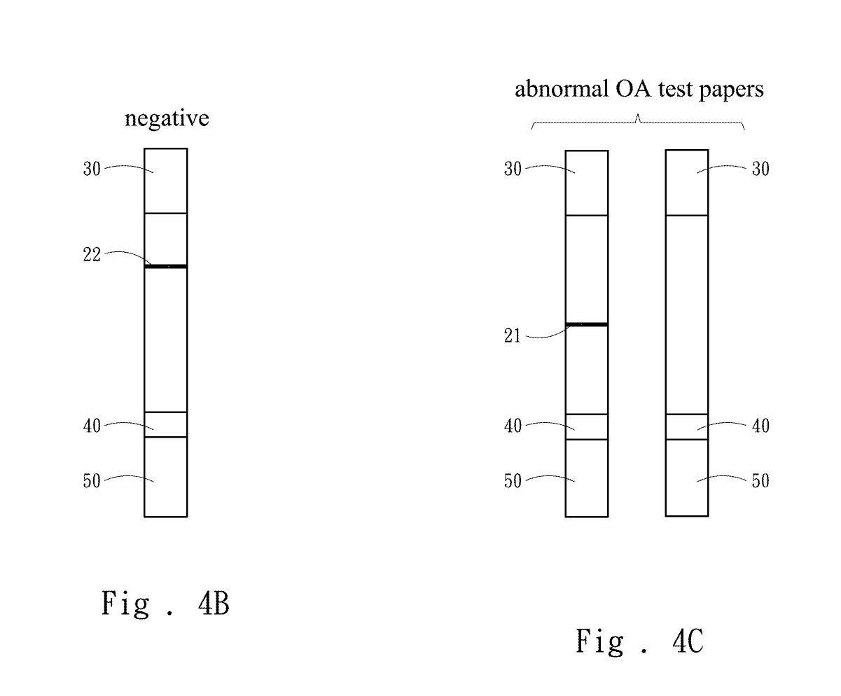

Refer to FIG. 1, FIG. 3 and FIGS. 4A-4C. The process and results undertaken by the instant osteoarthritis test paper are described below.

As shown in FIG. 4A, a drop of a urine sample 1 is dripped on the sample reception layer 50. The capillary action of the layers moves the sample toward the water absorption layer 30. If the urine sample 1 has COMP 2 specific to the OA patients, the COMP 2 will combine with the test complexes 41 in the antibody release layer 40 and will also combine with the monoclonal antibodies to COMP 211 in the result indication zone 21 to develop color. The residual test complexes 41 that do not combine with the COMP 2 will combine with the secondary antibodies 221 in the control zone 22 to develop color, whereby to guarantee normal operation of the instant OA test paper. As shown in FIG. 4B, if the urine sample 1 does not have the COMP 2, the color of the result indication zone 21 will not be developed, while only the control zone 22 will develop color as the test complexes 41 combine with the secondary antibodies 221, whereby to guarantee normal operation of the instant OA test paper.

As shown in FIG. 4C, in the event that the control zone 22 does not develop color, it means that no test complex 41 combines with the secondary antibodies 221 or indicates that other abnormal conditions occur. No matter whether the result indication zone 21 develops color, such a case indicates that the instant OA test paper is abnormal and that the test result is invalid.

In conclusion, the present invention has the following characteristics:
1. The present invention uses the combination of the polyclonal antibodies to COMP in the test complexes and the COMP in the urine sample to detect OA in the early stage, whereby to overcome the problem that the conventional X-ray inspection can only detect OA in the later stage or when the articular cartilage has been degenerated.
2. The instant OA test paper of the present invention realizes a non-invasive inspection of OA, and thus exempts testee from discomfort caused by the conventional invasive inspection methods.
3. The instant OA test paper of the present invention exempts from complicated analysis steps and is able to rapidly examine urine samples in easy steps to fast get the test results.

What is claimed is:

1. An instant osteoarthritis test paper, which is used to detect a urine sample, comprising:
a substrate;
a test result layer which is arranged on the substrate, and includes a result indication zone, a control zone, a test section between the result indication zone and the control zone, a first connection section arranged at one side of the control zone far away from the test section, and a second connection section arranged at one side of the result indication zone far away from the test section;
a water absorption layer connecting with the substrate and partially covering the first connection section;
an antibody release layer connecting with the substrate and partially covering the second connection section, wherein the antibody release layer includes a plurality of test complexes each including a colloidal gold and a plurality of polyclonal antibodies to COMP (Cartilage Oligomeric Matrix Protein) binding to the colloidal gold; and
a sample reception layer connecting with the substrate and partially covering the antibody release layer.

2. The instant osteoarthritis test paper according to claim 1, wherein the test result layer is water-absorptive nitrocellulose paper.

3. The instant osteoarthritis test paper according to claim 1, wherein the result indication zone includes a plurality of monoclonal antibodies to COMP, and the control zone includes a plurality of secondary antibodies.

4. The instant osteoarthritis test paper according to claim 1, wherein the water absorption layer and the test result layer are overlapped at an area having a width of 0.1-0.3 cm.

5. The instant osteoarthritis test paper according to claim 1, wherein the antibody release layer and the test result layer are overlapped at an area having a width of 0.1-0.3 cm.

6. The instant osteoarthritis test paper according to claim 1, wherein the antibody release layer is made of water-absorptive glass fiber.

7. The instant osteoarthritis test paper according to claim 1, wherein the sample reception layer is made of water-absorptive glass fiber.
